(19) United States
(12) Patent Application Publication
(10) Pub. No.: US 2020/0323635 A1
(43) Pub. Date: Oct. 15, 2020

(54) A DEVICE FOR INSERTING A GUIDE WIRE IN A BLOOD VESSEL

(71) Applicant: VIVHEART S.R.L., Milano (IT)

(72) Inventor: Matteo MONTORFANO, Milano (Ml) (IT)

(73) Assignee: VIVHEART S.R.L., Milano (IT)

(21) Appl. No.: 16/761,927

(22) PCT Filed: Jan. 9, 2019
(86) PCT No.: PCT/IB2019/050155

§ 371 Date: May 6, 2020

(30) Foreign Application Priority Data
Jan. 10, 2018 (IT) 102018000000671

(51) Int. Cl. A61F 2/24; A61F 2/95

(52) U.S. Cl. CPC A61F 2/2436; A61F 2/9517

(57) ABSTRACT

This disclosure provides a device having an outermost catheter, an intermediate catheter inserted into the outermost catheter, a innermost catheter inserted into the intermediate catheter, with different characteristics, configured to accurately control and reproducibly guide a wire through a heart valve.

### TECHNICAL FIELD

The present disclosure relates in general to catheters and more in particular to a device for inserting a guidewire into a blood vessel, wherein the device is self-centering and may be inserted into an artery for directing a guidewire through a heart valve.

### BACKGROUND OF THE INVENTION

Percutaneous valve replacement (Transcatheter Aortic Valve Replacement or more briefly TAVR) is a technique performed by inserting a catheter containing a guidewire, which is used as a guide for the implantation of a cardiac valve prosthesis. The catheter is threaded through the femoral artery and pushed up to the proximity of the defective heart valve or through the chest, for exiting the guidewire contained in the catheter near the cardiac valve so that it passes throughout it. Once the guidewire has passed throughout the heart valve, it is used to guide a valve prosthesis to the heart, where it must be positioned.

A difficulty related to this type of technique consists in the fact that the operator must be particularly skilled in order to quickly insert the guidewire through the faulty valve. The imaging equipment currently used in operating rooms only allow a two-dimensional view and not a three-dimensional view of the guidewire approaching the defective heart valve. As a consequence, from the two-dimensional image the operator may have the wrong impression of correctly directing the guidewire to the center of the heart valve, which is also narrow and degenerated, crooked, when instead the apical end of the guidewire is going to abut against the walls of the aorta.

In any case, even if a second apparatus for images arranged so as to provide a different view were available, there would be the problem of positioning and properly orienting a catheter while pushing forward the guidewire. Moreover, the guidewire is made of yielding material so as not to damage the tissues with which it comes into contact, so it bends easily even when it should remain straight to pass through the defective valve. This problem is even more pronounced in the so-called Valve-In-Valve technique (or more briefly VIV), in which a defective valve prosthesis is not completely removed, but is used as a frame in which a new percutaneous heart valve prosthesis is anchored. This new technique prevents the patient from the trauma of removing the old prosthetic valve, which remains attached to the heart tissue while the new valve prosthesis is attached to the old. Nevertheless this is critical because the guidewire, if pushed against the damaged portions of the old heart valve prosthesis, may cause the detachment of a part of it.

### SUMMARY

Studies performed by the applicant led to the design of a new device comprising multiple catheters with different characteristics, one inserted inside the other, to direct a guided thread through a blood vessel and/or a heart valve in a stable, carefully controlled and repeatable manner.

The device of this disclosure solves the problem thanks to an outermost catheter, to an intermediate catheter inserted into the outermost catheter and to a more internal catheter inserted into the intermediate catheter, each with its own peculiar characteristics. The intermediate catheter defines, at an apical portion, a tube on which it slides an anchoring element for anchoring to the inner walls of a blood vessel. The anchoring element comprises an annular slider sliding around the tube and a plurality of wire elements each having a proximal end attached to the tube and a distal end attached to the annular slider. The wire elements are made of a shape memory material and are configured to spontaneously bend away radially with respect to the tube to abut against the inner walls of the blood vessel at opposite points when they exit the outermost catheter, dragging the annular slider which is slid on the tube towards the proximal ends of the wire elements. The apical portion the innermost catheter is configured to assume a curved position at rest, and is configured to be elastically extended when inserted into the tube of the intermediate catheter and to spontaneously return to the curved position at rest as it exits from the tube.

Preferred or alternative embodiments are defined in the dependent claims.

The claims as filed are an integral part of this disclosure and are hereby incorporated by reference.

### BRIEF DESCRIPTION OF THE DRAWINGS

FIG. 1A is a front perspective view of a device of this disclosure for inserting a guidewire for heart valve prosthesis, in which the intermediate catheter and the inner catheter are in a retracted configuration with respect to the outer catheter.

FIG. 1B is a rear perspective view of the device of FIG. 1A, in which the rear hole is shown in the handle of the innermost catheter, through which the guidewire is inserted.

FIG. 2A shows the device of FIG. 1A, with the innermost catheter in a retracted configuration and the intermediate catheter in advanced configuration with respect to the outermost catheter, for expanding an anchoring element which abuts against the inner walls of a blood vessel.

FIG. 2B is a detailed view of the anchoring element of FIG. 2A, showing the slider of the anchoring element retracted around the tube of the intermediate catheter to bend the wire elements.

FIG. 3A shows the device of FIG. 2A, with the innermost catheter in an advanced configuration with respect to the outermost catheter to exit outside the intermediate catheter tube.

FIG. 3B is a detailed view of the anchoring element of FIG. 3A, showing the innermost catheter which spontaneously assumes a curved and orientable configuration when exiting from the intermediate catheter tube.

FIG. 4A shows the device of FIG. 3A, with the guidewire inserted through the innermost catheter.

FIG. 4B is a detailed view of the anchoring element of FIG. 4A, showing the guidewire oriented in the direction established by the innermost catheter.

### DETAILED DESCRIPTION

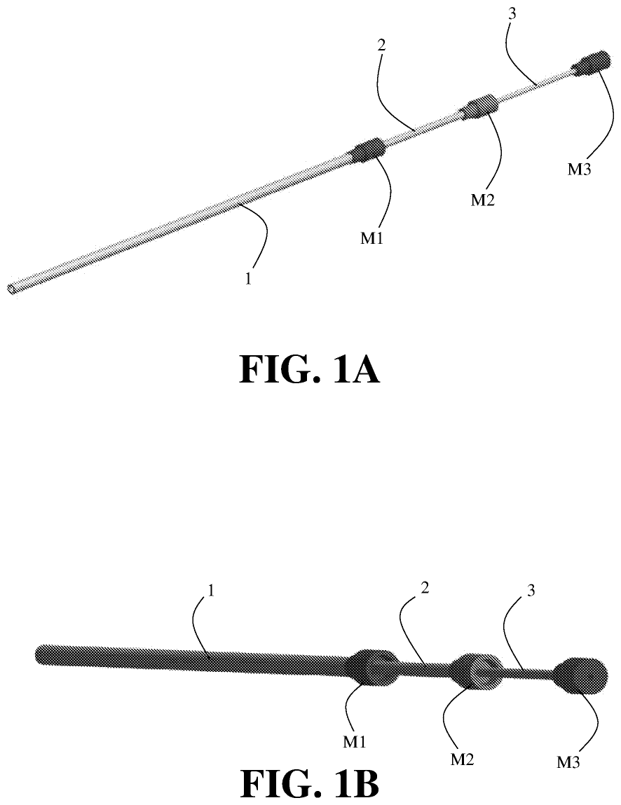

A device of this disclosure of a guidewire insertion through cardiac valve prosthesis is shown in FIGS. 1A and 1B. It is basically composed of three catheters inserted one inside the other: an outer catheter 1, an intermediate catheter 2 and an inner catheter 3. Conveniently, each of these three catheters may be rotated around its longitudinal axis and moved forward or backward by means of respective handles M1, M2 and M3. The three catheters 1, 2, 3, have different properties from each other and, thanks to the synergistic combination of these properties, allow the device of this disclosure to place a guidewire in a precise and easily reproducible way through a defective heart valve. All catheters are made of suitable material and have adequate length to be inserted into an artery of the human body. Typically, they are intended to be threaded into the femoral artery and to be pushed up to the aorta until they attain a defective heart valve. Alternatively, they may be placed at the chest of a patient in a position as close as possible to the defective heart valve.

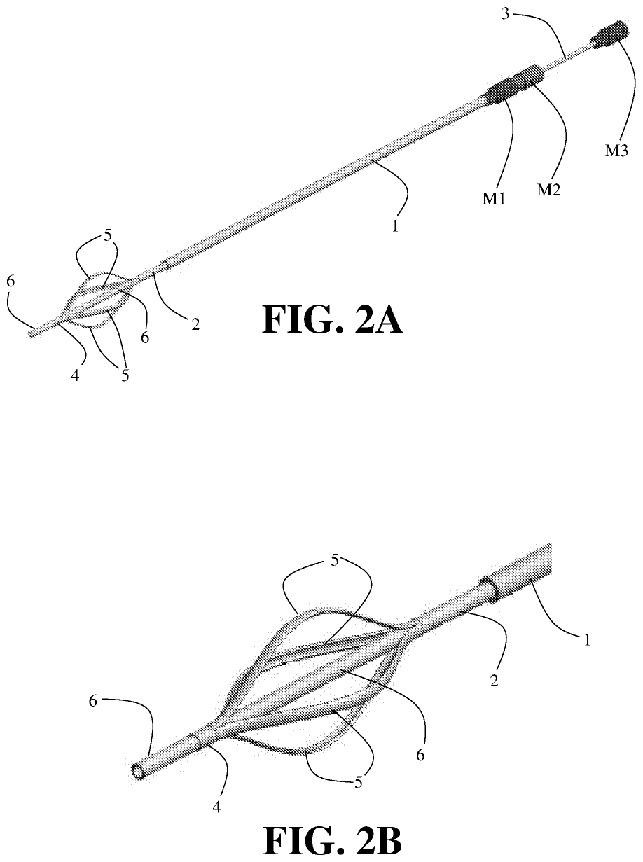

The outermost catheter 1 is a common catheter that may be inserted into an artery and its function is to convey the intermediate catheter 2 and the inner catheter 3, leaving them out only near the defective heart valve. When the apical end of the catheter 1 is near the defective heart valve, the handle M1 of the outer catheter 1 is retracted towards the handle M2 of the intermediate catheter 2, as shown in FIG. 2A.

The intermediate catheter 2 has, at its apical part, an anchoring element to the walls of a blood vessel. This anchoring element substantially comprises an annular slider 4, which connects wire elements 5 of a shape memory self-expandable frame and that slides freely on the tube 6 of the catheter 2.

The detailed view of FIG. 2B allows a better understanding of the structure of the self-expanding frame. As long as the intermediate catheter 2 is retracted into the outer catheter 1, the annular slider 4 is arranged around the tube 6 of the intermediate catheter 2 so that the wire elements 5 of the self-expanding frame are laying along the tube 6. When the handle M1 is retracted against the handle M2, the anchoring element protrudes from the outer catheter 1. The wire elements 5 are made of a shape-memory material, for example Nitinol, and tend to spontaneously bend as shown in FIG. 2B when free, allowing the ring slider 4 to slide along the tube 6 as in a mechanism for opening an umbrella. The length of the wire elements 5 of the self-expanding frame will be appropriately determined so that they bend and abut against the walls of the blood vessel in which the frame has expanded. In this way, the catheter 2 may be anchored where desired within the vessel, providing a reference point.

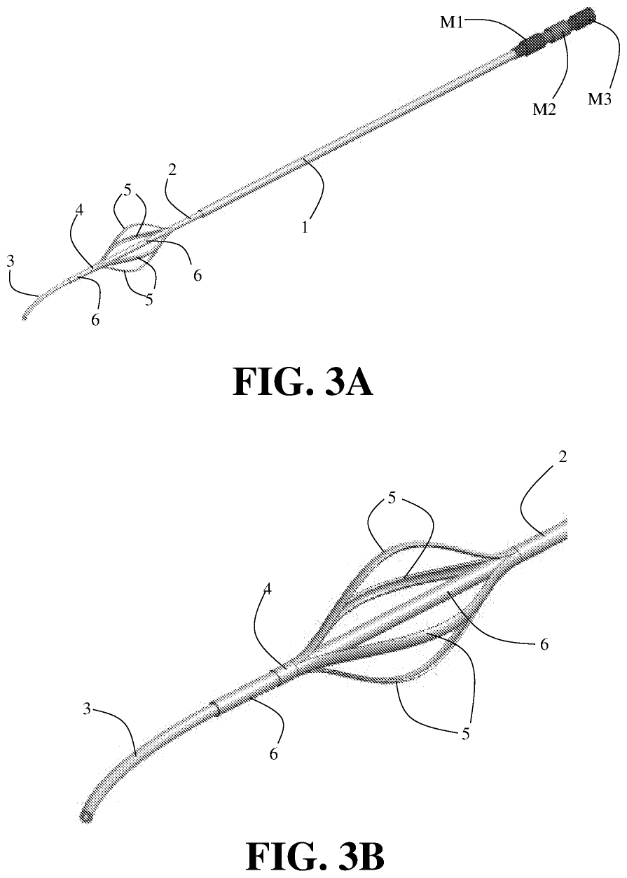

When the frame is against the blood vessel, the handle M3 is pushed towards the handle M2, as shown for example in FIG. 3A and in the detail view of FIG. 3B. The inner catheter 3 has at least one elastic apical part which, at rest, assumes a curved configuration.

When the apical part of the inner catheter 3 is inside the intermediate catheter 2, it is elastically forced to remain lying therein. As the handle M3 is pushed towards the handle M2, the apical part of the catheter 3 comes out of the tube 6 and the longer it protrudes from the tube 6 the more it bends. By pushing or retracting the handle M3 and/or by turning it on itself, the operator may adjust at will the curvature of the apical part of the catheter 3 and/or orient it in the blood vessel, in order to point it towards the center of the defective heart valve.

The intermediate catheter 2 is anchored to the walls of the blood vessel, thus the operator may easily maintain the orientation of the apical part of the catheter 3 by holding the M3 handle stationary for as long as desired. As a consequence, it is possible to use a unique imaging apparatus to verify the orientation of the apical portion of the catheter 3 according to a first point of view, then to move the same imaging apparatus to a different point of view to verify that the innermost catheter 3 is actually pointing at the center of the defective heart valve.

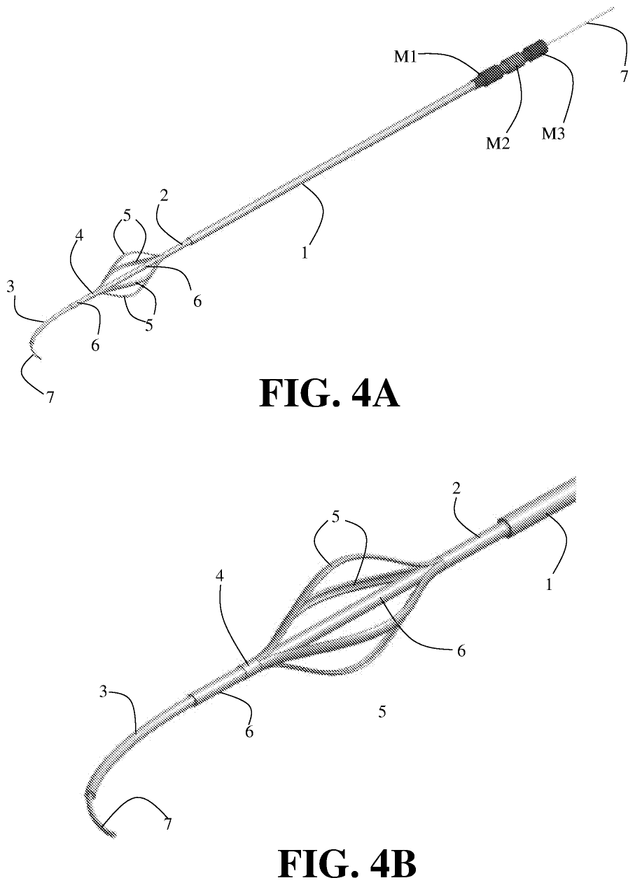

When the operator considers that the opening of the innermost catheter 3 is pointing precisely in the desired direction, it slides the guidewire 7 into the catheter 3 as shown in FIG. 4A and in the detail view of FIG. 4B. The guidewire 7, sliding in the catheter 3, may only be arranged in the desired direction since the first attempt, without any risk of damaging the walls of the blood vessel or of touching some delicate part of the defective heart valve.

Even the inner catheter 3, as well as the wire elements, may conveniently be made of a shape memory material, such as Nitinol.

Preferably, but not necessarily, the outermost catheter 1 will have a diameter not greater than 10 F (3.33 mm), in order to be inserted inside a femoral artery, and the lumen of the inner catheter 3 must be such as to contain a guidewire of at least 0.035 inches (0.89 mm). The lumen and the thickness of the walls of the intermediate catheter 2, as well as the thicknesses of the walls of the outermost catheter 1 and of the innermost catheter 3 will be easily determined accordingly by the expert technician to perform the functions illustrated above.

1. A device for inserting a guidewire suitable for hearth valve prosthesis in a blood vessel, comprising:
an outer catheter,
an intermediate catheter that may be inserted into said outer catheter,
an inner catheter that may be inserted in said intermediate catheter,
wherein said intermediate catheter has an apical portion that defines a tube onto which an anchoring element for anchoring to inner walls of a blood vessel slides, said anchoring element comprises an annular slider slidable around said tube and a plurality of wire elements each having a proximal end fixed to said tube and a distal end fixed to said annular slider, wherein said wire elements are made of a shape memory material and are configured to bend spontaneously for displacing radially away from said tube when they exit from said outer catheter, making said annular slider slide around the tube towards said proximal ends, said wire elements being configured to be in abutment against inner walls of the blood vessel at opposite points contrasting displacements of said tube;
wherein at least an apical portion of said inner catheter is configured to assume a curved shape at rest, said apical portion being configured to be elastically distended when inserted in the tube of said intermediate catheter and to assume spontaneously the curved shape at rest as far as it protrudes out of the tube.

2. The device according to claim 1, wherein:
the outer catheter has a respective first handle configured to push the outer catheter forward inside a blood vessel and to make said outer catheter slide on the intermediate catheter;
the intermediate catheter has a respective second handle configured to push forward and/or pull backward said intermediate catheter in said outer catheter;
the inner catheter has a respective third handle configured to push forward and/or pull backward said inner catheter in said intermediate catheter and to rotate it around a longitudinal axis, said third handle having a through hole to allow insertion of a guidewire from back.

3. The device according to claim 1, wherein said annular slider is integral with said wire elements in correspondence of their distal ends.

4. The device according to claim 1, wherein said wire elements are made of a shape memory material suitable for being put in contact with tissues of a human body.

5. The device according to claim 1, further comprising a wire guide for hearth valve prosthesis inserted in said inner catheter.

6. The device according to claim 1, wherein said wire elements and said apical portion of the inner catheter are made of Nitinol.
